(19) United States
(12) Patent Application Publication
(10) Pub. No.: US 2007/0276385 A1
(43) Pub. Date: Nov. 29, 2007

(54) Intramedullary Nail

(75) Inventors: Andre Schlienger, Basel (CH); Markus Buettler, Oensingen (CH); Peter Senn, Waldenburg (CH)

Correspondence Address: JONES DAY, 222 EAST 41ST STREET, NEW YORK, NY, 10017-6702, US

(73) Assignees: SYNTHES GMBH, OBERDORF (CH); SYNTHES (USA), WEST CHESTER, PA (US)

(21) Appl. No.: 10/590,125

(22) PCT Filed: Feb. 23, 2004
(86) PCT No.: PCT/CH04/00094

§ 371 Date: Jun. 4, 2007

(51) Int. Cl. A61B 17/58

(52) U.S. Cl. 606/72; 606/104; 606/60

(57) ABSTRACT

The intramedullary nail (1) is designated in particular for the tibia and has a distal end (2) for insertion into the medullary canal, a proximal end (3), a central axis (4) and a generally rod-like shape over the hole length L. The nail (1) has three distinct locking sections (5,6,7) with at least one through-hole (8) each for receiving locking screws whereby said three locking sections (5,6,7) are separated from each other by two distinct intermediate sections (9,10) having less through-holes (8) per length unit than each of the locking sections (5,6,7).

The invention relates to an intramedullary nail, in particular for the tibia, according to the preamble of claim 1.

Intramedullary nails with proximal and distal locking options are already known in the art. Depending on the fracture site and the anatomical situation, in particular for the tibia bone, an additional locking option on an intermediate level would be desirable. On this point, the invention intends to provide remedial measures. The invention is based on the objective of providing an intramedullary nail having an additional locking option, in the case of a tibial nail in the area of the Isthmus.

The invention solves the posed problem with an intramedullary nail that displays the features of claim 1.

The three level locking according to the invention optimizes the transmission of load by different distribution and transformation of the load applied to the nail. The additional intermediate locking level (between the proximal and distal level) serves as a support or artificial additional joint and therefore will divide the load on a new lever-arm-ratio. The intermediate level locking option may also serve in mechanical principle as an (elongated) Isthmus and will be the more efficient the bigger the gap between the nail diameter and the intramedullary canal of the bone is.

The various partial length of the intramedullary nail according to the invention are defined as follows:

L₅: proximal locking section extending from said proximal end to a distal boundary;

L₆: distal locking section extending from said distal end to a proximal boundary;

L₇: isthmus locking section located between said distal and proximal locking sections with a proximal boundary and a distal boundary;

L₉: distance between the proximal boundary of said isthmus locking section to said distal boundary of said proximal locking section.

L₁₀: distance of said distal boundary of said isthmus locking section to said proximal boundary of said distal locking section; and

L=(L₅+L₉+L₇+L₁₀+L₆ )

L_{D} distance between the distal end of the intramedullary nail to the through hole which is located nearest to said distal end.

Lₚ: distance between the proximal end of the intramedullary nail to the through hole which is located nearest to said proximal end.

In a special embodiment the distinct intermediate sections of the intramedullary nail have no through-holes. This feature increases the stability in the fractured area.

In a further embodiment the intramedullary nail exhibits the following features:

A) a proximal locking section extending from said proximal end over the distance 0,22 L<L₅<0,28 L in direction of said distal end and having a distal boundary;

B) a distal locking section extending from said distal end over the distance 0,18 L<L₆<0,22 L in direction of said proximal end and having a proximal boundary; and

C) an isthmus locking section located between said distal and proximal locking sections with a proximal boundary and a distal boundary and a length of 0,08 L<L₇<0,15 L. The advantage obtained by these features is the creation of enough space to place sufficient locking options in different directions.

In a further embodiment the proximal boundary of said isthmus locking section has a distance 0,27 L<L₉<0,33 L to said distal boundary of said proximal locking section. This enables to locate the locking section at the level of the isthmus where there is the strongest bone and the most narrow part of the bone; this means with other words: less deviation of the nail axis to the bone axis at this position.

In a further embodiment said distal boundary of said isthmus locking section has a distance of 0,13 L<L₁₀<0,30 L to said proximal boundary of said distal locking section. This feature allows to have a section free of locking holes where the surgeon can adjust the fracture line (in between the locking sections).

In a further embodiment the geometry of the intramedullary nail obeys to the condition: 0,32 L<(L₁₀+L₆)<0,50 L. That guarantees that the isthmus or intermediate locking section is in the area of the isthmus well away from the spongy bone.

In a further embodiment the intramedullary nail has a first intermediate section having the length L₉ between said proximal locking section and said isthmus locking section and which preferably has no through holes. This feature increases the strength of the intramedullary nail.

In a further embodiment the intramedullary nail has a second intermediate section between said distal locking section and said isthmus locking section having the length L₁₀ and which preferably has no through holes. This measure further increases the strength of the intramedullary nail.

In a further embodiment the isthmus locking section has two through holes, preferably arranged at a relative angle α in the range of 60°<α<120°. This leads to a reduction of some degrees of freedom in the antero-posterior and medio-lateral direction.

In a further embodiment the through hole which is located nearest to said distal end of said intramedullary nail has a distance L_{D} to said distal end in the range of 0,01 L<L_{D}<0,38 L.

In another embodiment the through hole which is located nearest to said proximal end has a distance L_{P} to said proximal end in the range of 0,01 L<L_{P}<0,70 L.

In a further embodiment said proximal locking section having the length L₅ and said first intermediate section having the length L₉ are arranged at an angle β in the range of 7°<β<13°. This configuration allows an easier insertion of the intramedullary nail into the bone and a proper alignment of the nail axis to the bone axis (biomechanical axis).

The invention and additional configurations of the invention are explained in even more detail with reference to the partially schematic illustration of several embodiments.

The invention is described below on the basis of advantageous embodiments and with reference to the attached drawings in which:

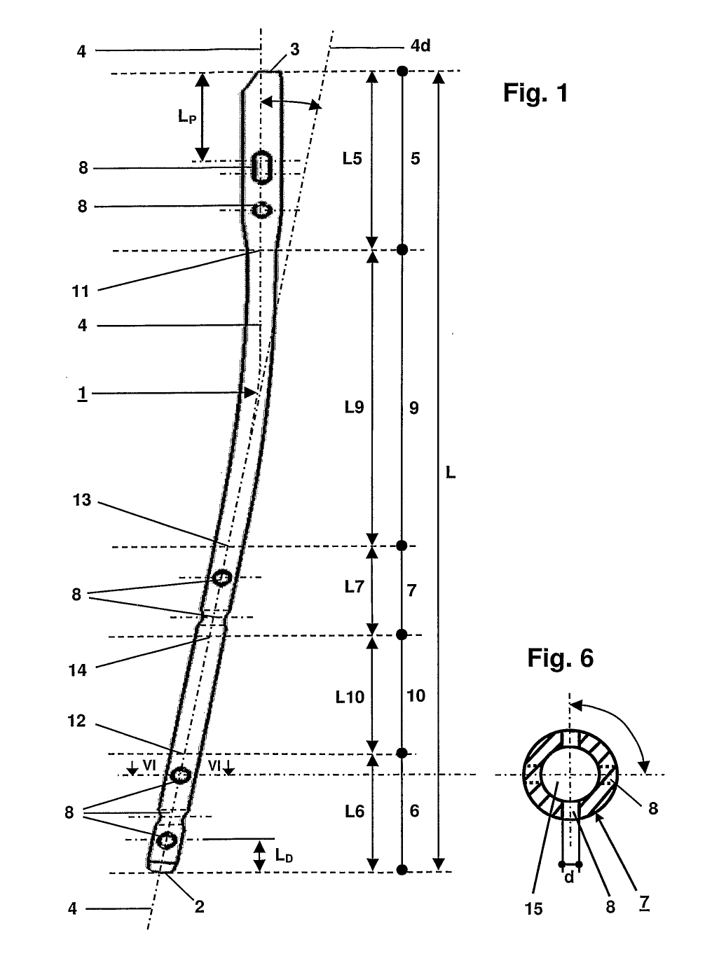

FIG. 1 shows, in a longitudinal view, an intramedullary nail according to the invention;

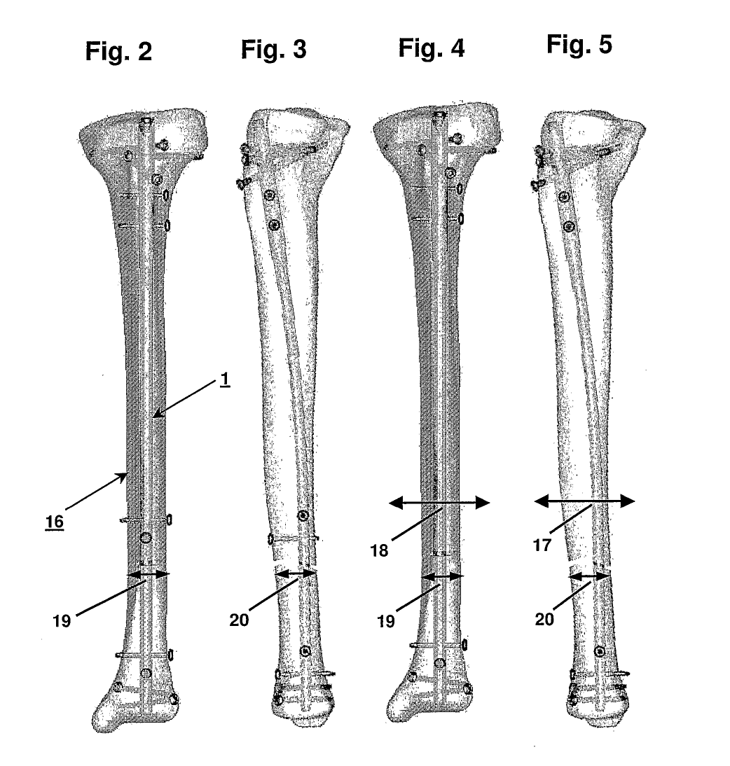

FIG. 2 shows an antero-posterior (or frontal) view of a modified intramedullary nail according to the invention inserted in a schematically represented tibia bone;

FIG. 3 shows the intramedullary nail of FIG. 2 in a latero-medial (or sagittal) view;

FIG. 4 shows an antero-posterior (or frontal) view of an intramedullary nail according to prior art with only two level locking;

FIG. 5 shows the intramedullary nail according to prior art of FIG. 4 in a latero-medial view; and

FIG. 6 shows an orthogonal section of the nail according to FIG. 1 along the line VI-VI in the region of one of the locking holes.

The intramedullary nail 1 shown in FIG. 1 has a distal end 2 for insertion into the medullary canal, a proximal end 3, a central axis 4 and a generally rod-like shape over the hole length L. The intramedullary nail 1 has three distinct locking sections 5,6,7. Each of these locking sections 5,6,7 has a number of through-holes 8 for receiving locking screws. Proximal locking section 5 has two through holes 8, one of which is an elongated one. Distal locking section 6 has three trough holes 8 two of which are parallel and the intermediate third through hole is running vertical to the other two. As shown in FIG. 6 the isthmus locking section 7 has two through holes 8 which are disposed at an angle α of 90°.

Said three locking sections 5,6,7 are separated from each other by two distinct intermediate sections 9,10 having no through-holes 8.

The proximal locking section 5 extends from said proximal end 3 of said intramedullary nail 1 over a distance L₅ equal to 0,25 L in direction of said distal end 2 to a distal boundary 11. The distal locking section 6 extends from said distal end 2 over the distance L₆ equal to 0,2 L in direction of said proximal end 3 to a proximal boundary 12 and the isthmus locking section 7 located between said distal and proximal locking sections 5,6 with a proximal boundary 13 and a distal boundary 14 has a length of L₇ equal to 0,115 L. Said proximal boundary 13 of said isthmus locking section 7 has a distance L₉ equal to 0,3 L to said distal boundary 11 of said proximal locking section 5. Said distal boundary 14 of said isthmus locking section 7 has a distance L₁₀ equal to 0,215 L to said proximal boundary 12 of said distal locking section 6. Therefore the sum of the two distances L₁₀+L₆ is equal to 0,415 L.

The intramedullary nail 1 further has a first intermediate section 9 having the length L₉ between said proximal locking section 5 and said isthmus locking section 7 and which has no through holes 8. It further has a second intermediate section 10 between said distal locking section 6 and said isthmus locking section 7 having the length L₁₀ and which has no through holes 8.

The through hole 8 which is located nearest to said distal end 2 has a distance L_{D} to said distal end 2 which is equal at least the diameter “d” of said through hole 8.

Said proximal locking section 5 having the length L₅ and said first intermediate section 9 having the length L₉ are arranged at an angle β equal to 100 being the angle between the prolongation 4d of the central axis 4 in the distal locking section 6 and the central axis 4 in the proximal locking section 5.

As shown in FIG. 6 the intramedullary nail 1 has a cannulation 15 coaxial to the central axis 4.

FIGS. 2 and 3 show the intramedullary nail 1 according to the invention implanted in a tibia bone with a distal fracture. In FIG. 2 the intramedullary nail 1 is shown in the antero-posterior view with several locking screws at three distinct levels. The fracture line is below the isthmus locking level. The same situation is represented in FIG. 3 in a sagittal view. Arrows 19 and 20 in FIGS. 2 and 3 indicate the range of freedom for the distal bone fragment of the treated bone which is similar to the degree of freedom of a conventional intramedullary nail (FIG. 4 and 5) whereas the proximal fragment has a reduced freedom compared to a conventional nail due to its isthmus locking section. If the fracture line is above the isthmus section the distal fragment has a reduced degree of freedom.

FIGS. 4 and 5 show a conventional intramedullary nail with no isthmus locking section. FIG. 4 represents an antero-posterior view and FIG. 5 a sagittal view. Arrows 17 and 18 in FIGS. 4 and 5 indicate the range of freedom for the proximal bone fragment of the treated bone without an isthmus locking section which is much larger compared to the intramedullary nail 1 according to the invention represented in FIGS. 2 and 3.

1-11. (canceled)

12. An intramedullary nail comprising:
an elongated nail body having a proximal end, a distal end for insertion into the medullary canal, a central axis and a total length L;
three locking sections along the length of the nail body between the proximal and distal ends, each locking section including a through-hole for receiving a locking screw; and
two distinct intermediate sections separating the three locking sections, the intermediate sections having fewer through-holes per unit length than each of the three locking sections,
wherein the locking section nearest the proximal end has a length L₅ forming an angle β with the intermediate section adjacent the proximal locking section where β is in the range of 7°<β<13°.

13. The intramedullary nail of claim 12, wherein the intermediate sections have no through-holes

14. The intramedullary nail of claim 12, wherein the three locking sections include:
a proximal locking section extending from the proximal end toward the distal end over a length L₅, where 0.22 L<L₅<0.28 L, and having a distal boundary;
a distal locking section extending from the distal end toward the proximal end over a length L₆, where 0.18 L<L₆<0.22 L, and having a proximal boundary; and
an isthmus locking section located between the distal and proximal locking sections, and having a proximal boundary, a distal boundary and a length L₇ where 0.08 L<L₇<0.15 L.

15. The intramedullary nail of claim 14, wherein the proximal boundary of the isthmus locking section is spaced a distance L₉ from the distal boundary of the proximal locking section, where 0.27 L<L₉<0.33 L.

16. The intramedullary nail of claim 14, wherein the distal boundary of the isthmus locking section is spaced a distance L₁₀ from the proximal boundary of the distal locking section, where 0.13 L<L₁₀<0.30 L.

17. The intramedullary nail of claim 16, where 0.32 L<(L₁₀+L₆)<0.50 L.

18. The intramedullary nail of claim 14, wherein the first intermediate section has a length L₉ between the proximal locking section and the isthmus locking section, and the first intermediate section has no through holes.

19. The intramedullary nail of claim 18, wherein the second intermediate section has a length L₁₀ between the distal locking section and the isthmus locking section, and the second intermediate section has no through holes.

20. The intramedullary nail of claim 14, wherein the isthmus locking section includes two through holes arranged at a relative angle α in the range of 60°<α<120°.

21. The intramedullary nail of claim 1, wherein the through hole located nearest to the distal end is spaced a distance L_{D} to the distal end, where 0.01 L<L_{D}<0.38 L.

22. The intramedullary nail of claim 1, wherein the through hole located nearest to the proximal end is spaced a distance L_{P} to the proximal end, where 0.01 L<L_{P}<0.70 L.

23. An intramedullary nail comprising:
an elongated nail body having a proximal end, a distal end for insertion into the medullary canal, a central axis and a total length L;
a proximal locking section, distal locking section, and isthmus locking section spaced along the length of the nail body, the proximal locking section nearest the proximal end, the distal locking section nearest the distal end, and the isthmus locking section located between the proximal and distal locking sections, and each locking section including a through-hole for receiving a locking screw;
a first intermediate section separating the proximal and isthmus locking sections, and a second intermediate section separating the isthmus and distal locking sections, each intermediate sections having fewer through-holes per unit length than the locking sections,
wherein the proximal locking section forms an angle β with the first intermediate section, where β is in the range of 7°<β<13°.

24. The intramedullary nail of claim 23, wherein the intermediate sections have no through-holes.

25. The intramedullary nail of claim 23, wherein the isthmus locking section includes two through holes arranged at a relative angle α in the range of 60°<α<120°.
